(19) United States
(12) Patent Application Publication
(10) Pub. No.: US 2012/0316319 A1
(43) Pub. Date: Dec. 13, 2012

(54) ARYLETHYNYLXANTHENE DYES

(76) Inventors: Alexey Ustinov, Moscow (RU); Vladimir Korshun, Moscow (RU)

(21) Appl. No.: 13/492,888

(22) Filed: Jun. 10, 2012

Related U.S. Application Data
(60) Provisional application No. 61/495,511, filed on Jun. 10, 2011.

(51) Int. Cl. C09B 1/00; C08G 63/48; C07H 1/00; C07H 21/00; C07K 14/00; C07K 16/00

(52) U.S. Cl. 530/350; 530/391.3; 536/1.11; 536/22.1; 552/271; 554/1

(57) ABSTRACT

Disclosed are 9-arylethynylxanthenes which possess useful fluorescent properties, and can be used as fluorescent dyes, and labels.

This patent application claims the benefit of U.S. Patent Provisional Application No. 61/495,511 filed on Jun. 10, 2011 and is incorporated herein by reference.

### BACKGROUND OF THE INVENTION

This invention is related to new class of fluorescent dyes—9-arylethynylxanthenes, which can be used for the labeling of various biomolecules, fluorescent staining, NIR imaging, in laser dyes, and also have other applications based on fluorescence.

Fluorescent dyes are molecules capable of absorbing energy of excitation radiation, and emit it as fluorescence. Ability of molecule to fluoresce in UV, visible, and IR range depends on molecular structure. Only a limited number of compound classes can be used as fluorescent labels, because useful fluorescent labels should have high quantum yield of fluorescence, high extinction coefficients at excitation wavelength, as well as chemical stability, and photostability.

Most commonly used fluorescent dyes belong to cyanines, fluoresceins, and rhodamines. Fluoresceins and rhodamines are xanthene dyes. Other common classes are coumarines and polyaromatic hydrocarbons (PAHs). Despite wide use, existing dyes have disadvantages. Most PAH and courmarines are only blue- and UV-emitting fluorophores. Cyanines generally have lower fluorescence quantum yields than fluoresceines and rhodamines. It is difficult to design fluoresceins and rhodamines emitting in red and infrared wavelengths.

Although fluoresceins and rhodamines are the most widely used xanthenes, there are examples of other 9-arylxanthenes. Fluorescein analogs bearing no 2-carboxylic group are known, such as Tokyo Green (Urano, Y.; Kamiya, M.; Kanda, K.; Ueno, T.; Hirose, K.; Nagano, T. J. Am. Chem. Soc., 2005, 127, 4888-4894), and Pennsylvania Green (Mottram, L. F.; Boonyarattanakalin, S.; Kovel, R. E.; Peterson, B. R. Org. Lett., 2006, 8(4), 581-584; Mottram, L. F.; Maddox, E.; Schwab, M.; Beaufils, F.; Peterson, B. R. Org. Lett., 2007, 9(19), 3741-3744).

Arylxanthene analogs of rhodamines were prepared from xanthones (Wu, L.; Burgess, K. J. Org. Chem., 2008, 73, 8711-8718). Fluorescent properties of the products were determined. Fluorescence maxima for these dyes varied in narrow range of 580-606 nm.

Xanthenes bearing no aryl substituents at 9-position are rare and relatively unexplored. 9-Amino- and 9-thiopyronin dyes are known (Wu, L.; Burgess, K. Org. Lett., 2008, 10(9), 1779), as well as 9-alkyl (U.S. Pat. No. 5,332,666; Amat-Guerri, F.; Martin, M. E.; Martinez-Utrilla, R.; Pascual, C.; J. Chem. Res., 1988, 6, 1447-1465), and 9-cyano (not isolated; C. D. Ritchie, C. Kubisty, G. Y. Ting, J. Am. Chem. Soc., 1983, 105(2), 279-284).

The invention discloses synthesis and fluorescent properties of yet unknown 9-arylethynyl xanthenes which possess red-shifted fluorescence, high quantum yields, and other useful spectral properties.

### BRIEF DESCRIPTION OF DRAWINGS

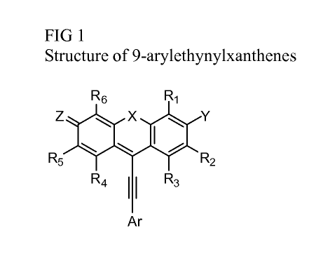

(FIG. 1) Generic structure of 9-arylethynylxanthenes

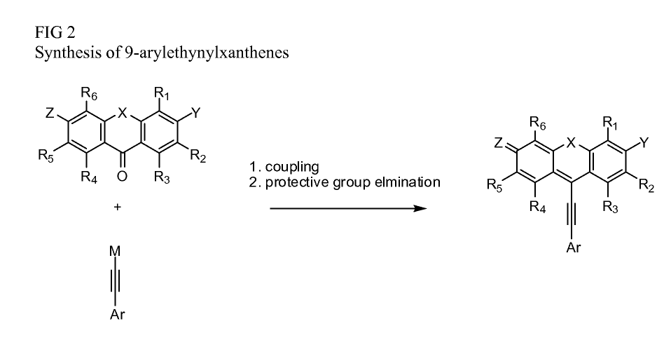

(FIG. 2) Scheme of synthesis of 9-arylethynylxanthenes

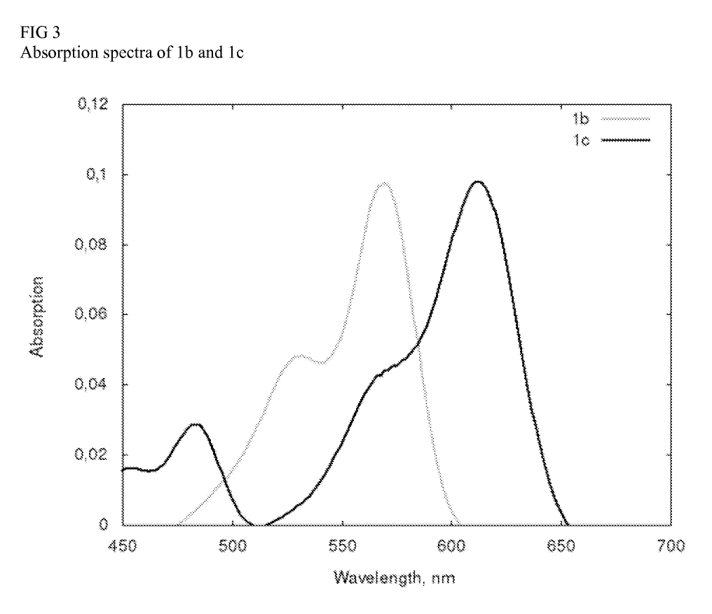

(FIG. 3) Absorption spectra of 1b and 1c

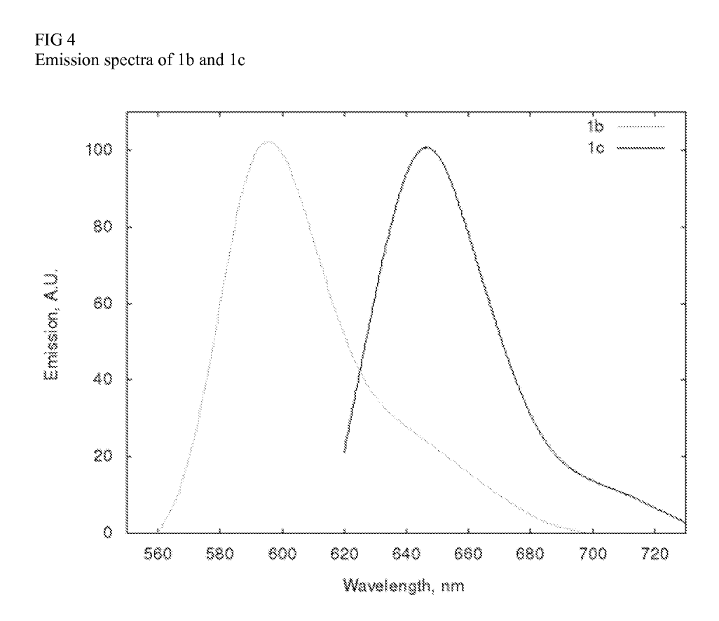

(FIG. 4) Fluorescence emission spectra of 1b and 1c

### DESCRIPTION OF THE INVENTION

In this patent application, we disclose a new class of fluorescent dyes which are yet unknown structural derivatives of xanthene (formula A).

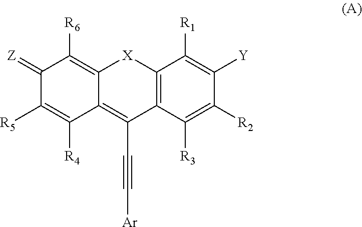

Where Z is chosen among O, and N⁺RR′, where R and R′ are alkyls, Y is chosen among OH, and NRR′, where R₁,R₂,R₃,R₄,R₅,R₆ are independently selected of alkyl, aryl, alkyloxy, aryloxy, halogen, SO₃H, SO₂R, S(O)R, CO₂H, CO₂R, CN, CONR₂, PO₂OH, neutral groups that reduce water solubility, polar groups that increase water solubility, functional groups that can be used in labeling reactions, reactive groups, electron donating and withdrawing groups that shift the absorption and emission wavelengths of the fluorescent molecule, lipid and hydrocarbon solubilizing groups, hydrogen, aryl, heteroaryl, cyano, nitro, aldehyde, halogen, hydroxy, amino, quaternary amino, acetal, ketal, phosphoryl, sulphydryl, water-solubilizing groups, and alkyl groups optionally substituted by amino, C1-C20 alkyl-substituted amino, quaternary amino, carbonyl including aldehyde and ketone, acetal, ketal, halo, cyano, aryl, heteroaryl, hydroxyl, sulphonate, sulphate, carboxylate, amide, nitro, and groups reactive with amino, hydroxyl, aldehyde, phosphoryl, or sulphydryl groups, Ar is aryl or heteryl, X is oxygen, sulfur, amino, sulfoxide, sulfone, selenium, tellurium, Y and Z selected independently of hydroxyl, unsubstituted, mono- or di-substituted amino, thiol, alkyl- or arylsulfide.

The synthesis of the dyes can be accomplished using generic scheme (FIG. 2) involving reaction of protected substituted xanthone with metal arylacetylide or arylacetylide derivative generated in situ.

The scheme described allows to obtain arylethynylxanthenes in synthetically useful yields.

Fluorescence and absorption spectra of 9-arylethynylxanthenes were determined. The spectra show pronounced red shift compared to analogous fluoresceins, and rhodamines. Compounds possess high fluorescence quantum yields.

Absorption and fluorescent spectra of the compounds were determined (table 1).

TABLE 1
Spectral properties of compounds of formula A (R₁ . . . R₆ = H).

| | | Absorption | Emission | Quantum |
|---|---|---|---|---|
| Compound | Radicals (formula A) | maximum, nm | maximum, nm | yield |
| 1a | Y = OH, Z = O, Ar = Ph | 570 | 597 | 0.84 |
| 1b | Y = OH, Z = O, Ar = Mes | 572 | 599 | 0.91 |
| | Reference fluorescein analog: Tokyo Green | 491 | 510 | |
| | (4, Y = O, Z = OH, Ar = o-tolyl) | | | |
| | (data from: Urano, Y.; Kamiya, M.; Knada, K.; | | | |
| | Ueno, T.; Hirose, K.; Nagano, T. J. Am. Chem. | | | |
| | Soc., 2005, 127, 4888-4894) | | | |
| 1c | Y = Z = morpholine, Ar = Mes, chloride | 612 | 647 | 0.93 |
| | Reference rhodamine analog 4, | 556 | 582 | |
| | Y = Z = morholine, Ar = phenyl, chloride form | | | |
| | (data from: Wu, L.; Burgess, K. J. Org. Chem., | | | |
| | 2008, 73, 8711-8718) | | | |

The results show that introduction of arylethynyl group in xanthene dyes leads to strong red shift in absorption and fluorescence of the dyes, compared to similar structures possessing no arylethynyl moiety. In case of fluoresceins, new dyes show approximately 80 nm shift in both absorption and fluorescence compared to reference 9-arylxanthenes. In case of rhodamine analogs, new compound show approximately 60 nm shift in absorption and fluorescence, with some Stokes shift increase.

The data show that introduction of arylethynyl moiety leads to significant change of fluorescent properties, allowing to use these compounds as higly valuable fluorescent probes.

The following examples describe syntesis of specific compounds of formula A.

### EXAMPLES

500 MHz ¹H and 125.7 MHz ¹³C NMR spectra were recorded on a Bruker DRX-500 spectrometer and referenced to CDCl3 (7.25 ppm) and DMSO-d6 (2.50 ppm). Analytical thin-layer chromatography was performed on the Kieselgel 60 F254 precoated aluminium plates (Merck), spots were visualised under UV light (254 nm). Column chromatography was performed on silica gel (Merck Kieselgel 60 0.040-0.063 mm). Reagents obtained from commercial suppliers were used as received. Solvents were mainly HPLC grade and used without further purification unless otherwise noted. THF was distilled over powdered LiAlH4 or over sodium benzophenone ketyl and stored over 4 Å molecular sieves under nitrogen. DMF was freshly distilled under reduced pressure. 3,6-Bis(N-morpholino)xanth-9-one was prepared according as described in Wu, L.; Burgess, K. J. Org. Chem., 2008, 73, 8711-8718.

Fluorescence was measured using Varian Cary Eclipse instrument. Absorption spectra were recorded with Varian Cary 100 UV/Vis spectrophotometer.

### Example 1

### 3,6-Bis(dimethyl-tert-butylsilyloxy)xanth-9-one.

A mixture of solid 3,6-dihydroxyxanth-9-one (1.14 g, 5 mmol) and imidazole (1.73 g, 11.5 mmol) was dissolved in dry dimethylformamide (12 mL), and solid chlorodimethyl-tert-butylsilane (1.02 g, 15 mmol) was added. The reaction is stirred for 30 minutes at room temperature (full dissolution followed by precipitation of product takes place), and water (100 mL) is then added. The mixture is then mixture is stirred for 15 minutes, and precipitated product is filtered off. The precipitate is washed with water (3×60 mL), dried on air, and in vacuum to give 2.22 g (97%) of pure title compound as white powder.

9-Phenylethynyl-3,6-dihydroxyxanthene (1a).

Phenylacetylene (220 μL, 2 mmol) was dissolved in dry toluene (4 mL) under argon. Butyllithium (700 μL of 2.5 M solution in hexanes, 1.4 mmol) was slowly added via syringe during vigorous stirring. The mixture was stirred for 10 min at room temperature, and then added to a solution of 3,6-bis(dimethyl-tert-butylsilyloxy)xanth-9-one (456 mg, 1 mmol) in toluene (4 mL). The reaction was stirred for 40 minutes at room temperature, and evaporated in vacuum. The residue was dissolved in THF (6 mL), and triethylamine trihydrofluoride (245 μL, 1.5 mmol) was added. The mixture was stirred for 30 minutes, and evaporated. The residue was dissolved in CHCl₃-MeOH (10:1), and subjected to column chromatography on silica gel (eluent 5% to 15% MeOH in CHCl₃, then 0.5% AcOH+15% MeOH in CHCl₃). Fractions containing title compound were pooled, and evaporated. The residue was co-evaporated with ether (2×50 mL), and dried in vacuum to give 220 mg (70%) of pure title compound as black crystals. R_{f} 0.49 (15% MeOH in CHCl₃).

### EXAMPLE 2

9-(2,4,6-trimethylphenyl)-3,6-dihydroxyxanthene (1b).

Mesitylacetylene (288 mg, 2 mmol) was dissolved in dry toluene (4 mL) under argon. Butyllithium (700 μL of 2.5 M solution in hexanes, 1.4 mmol) was slowly added via syringe during vigorous stirring. The mixture was stirred for 20 min at room temperature, and then added to a solution of 3,6-bis(dimethyl-tert-butylsilyloxy)xanth-9-one (456 mg, 1 mmol) in toluene (4 mL). The reaction was stirred for 4 hours at 60° C., cooled, and evaporated in vacuum. The residue was dissolved in THF (6 mL), and triethylamine trihydrofluoride (2454, 1.5 mmol) was added. The mixture was stirred for 30 minutes, and evaporated. The residue was dissolved in CHCl₃-MeOH (10:1), and subjected to column chromatography on silica gel (eluent 10% MeOH and 1% AcOH in CHCl₃ to 10% AcOH and 40% MeOH in CHCl₃). Fractions containing title compound were pooled, and evaporated. The residue was co-evaporated with ether (2×50 mL), and dried in vacuum to give 240 mg (68%) of pure title compound as black crystalls. R_{f} 0.21 (15% MeOH in CHCl₃).

### EXAMPLE 3

9-(2,4,6-trimethylphenyl)-3,6-dimorpholinoxanthenium chloride (8c).

Mesitylacetylene (288 mg, 2 mmol), and N,N,N′, N′-tetramethylethylenediamine (210 μL, 1.4 mmol) were dissolved in dry toluene (4 mL) under argon. Butyllithium (700 μL of 2.5 M solution in hexanes, 1.4 mmol) was slowly added via syringe during vigorous stirring. The mixture was stirred for 20 min at room temperature, and solid 3,6-bis(N-morpholino)xanth-9-one (366 mg, 1 mmol). The reaction was stirred for 4 hours at 70° C., cooled, and poured into a mixture of 30 mL ethanol, 20 mL of water, and 1 mL of 10 M aqueous HCl. The mixture was cooled to −20° C. The title compound precipitated and was filtered off to give 450 mg (85%) of pure title compound as dark purple crystals.

1. Compounds of formula A.

2. Compounds of claim 1 where R₁,R₂,R₃,R₄,R₅,R₆ are independently selected of alkyl, aryl, alkyloxy, aryloxy, halogen, SO3H, SO2R, S(O)R, CO2H, CO2R, CN, CONR2, PO2OH, neutral groups that reduce water solubility, polar groups that increase water solubility, functional groups that can be used in labeling reactions, reactive groups, electron donating and withdrawing groups that shift the absorption and emission wavelengths of the fluorescent molecule, lipid and hydrocarbon solubilizing groups, hydrogen, aryl, heteroaryl, cyano, nitro, aldehyde, halogen, hydroxy, amino, quaternary amino, acetal, ketal, phosphoryl, sulphydryl, water-solubilizing groups, and alkyl groups optionally substituted by amino, C1-C20 alkyl-substituted amino, quaternary amino, carbonyl including aldehyde and ketone, acetal, ketal, halo, cyano, aryl, heteroaryl, hydroxyl, sulphonate, sulphate, carboxylate, amide, nitro, and groups reactive with amino, hydroxyl, aldehyde, phosphoryl, or sulphydryl groups, Ar is aryl or heteryl, X is oxygen, sulfur, amino, sulfoxide, sulfone, selenium, tellurium, Y and Z selected independently of hydroxyl, unsubstituted, mono- or di-substituted amino, thiol, alkyl- or arylsulfide.

3. Fluorescent labeled target material comprising a target material selected from the group consisting of antibody, lipid, protein, carbohydrate, nucleotides which contain or are derivatized to contain one or more of an amino, sulphydryl, carbonyl, hydroxyl and carboxyl, phosphate and thiophosphate groups, and oxy or deoxy polynucleic acids which contain or are derivatized to contain one or more of an amino, sulphydryl, carbonyl, hydroxyl and carboxyl, phosphate and thiophosphate groups, microbial materials, drugs, toxins, particles, plastics or glass surfaces and polymers covalently bound to a compound according to claim 1.

4. Method for the production of compounds according to claim 1 utilizing coupling of metal acetylide or in situ generated acetylide anion with xanthone.
